(19) United States
(12) Patent Application Publication
(10) Pub. No.: US 2017/0128632 A1
(43) Pub. Date: May 11, 2017

(54) DEVICE AND METHOD FOR CONTROLLING THE RELEASE OF BIOACTIVE AND THERAPEUTIC AGENTS FROM AN IMPLANTABLE MEDICAL DEVICE

(71) Applicant: William Charles McJames, II, Hillsborough, NJ (US)

(72) Inventor: William Charles McJames, II, Hillsborough, NJ (US)

(21) Appl. No.: 14/936,727

(22) Filed: Nov. 10, 2015

(51) Int. Cl. A61L 31/16; A61L 31/04; A61F 2/00; A61L 31/14

(52) U.S. Cl. CPC A61L 31/16; A61L 31/148; A61L 31/041; A61F 2/0063; A61F 2/0077; A61L 2300/402; A61L 2300/604

(57) ABSTRACT

An implantable medical device for controlling the rate of release of a therapeutic agent is provided. The implantable medical device features at least one non-bioresorbable polymer, and/or at least one bioresorbable polymer, where the bioresorbable polymers have different degrees of hydrophilicity. Further, the implantable medical device incorporates at least one therapeutic agent contained in said at least one of non-bioresorbable polymer and/or said at least one of bioresorbable polymer. Preferably, the therapeutic agent is at least one anesthetic.

### CLAIM OF PRIORITY

This application does not claim priority to any patent or patent application.

### FIELD OF THE EMBODIMENTS

The present invention and its embodiments relate to a device and corresponding method for controlling the release of bioactive and therapeutic agents from an implantable medical device. In particular, the present invention and its embodiments relate to an implantable mesh infused with anesthetics.

### BACKGROUND OF THE EMBODIMENTS

Local delivery of medications at a specific anatomical site is preferable to systemic administration for several reasons, including efficacy at lower doses, and avoidance of drug side effects. Methods for delivering medications are well described in the literature, including injecting microspheres or similar particles, applying topical gels, implanting polymeric drug depots, implanting films, meshes or similar drug delivery devices, and other means familiar to those skilled in the art. The preferred method of delivering drugs to a surgical site is to incorporate them into a device already being used at the site as a part of a surgical procedure.

Products such as stents and balloons coated with drugs that retard intimal hyperplasia of blood vessels, catheters and cannula infused with antibiotics to prevent infection via central venous lines, and drug depots for various compositions such as antibiotics, anesthetics, anti-inflammatories, wound healing agents, hormones, growth factors and the like are examples of such devices described in the literature. Methods of incorporating the therapeutic agents into a reservoir or depot, or into a medical device itself include: chemical and electrostatic bonding or grafting, coating, absorption, adsorption, admixing, blending, lamination and impregnation. Polymeric and biologic materials have been used as reservoirs or depots for therapeutic agents wherein the reservoirs or depots can be in the form of capsules, microspheres, microparticles, microcapsules, microfibers, particles, granules, nanospheres, coatings, matrices, wafers, pills pellets, emulsions, liposomes, micelles, sheets, strips, fibers, mesh, films, and the like, or some combination of these forms. Reservoirs and depots can be attached to or incorporated into a medical device, or alternately, the device itself may have a therapeutic effect.

Controlling the rate of release of the therapeutic agent from the reservoir, depot or medical device is an important requirement in many medical applications. There are generally three basic methods for incorporating drugs into an implantable medical device, and releasing the drugs from said device in a controlled manner. The first method involves release from a porous reservoir, such as open cell foam, or materials such as polyethylene and polyurethane, homopolymers such as poly(n-butyl methacrylate) and copolymers such as poly(ethylene-co-vinyl acetate) and poly(isobutylene-co-styrene) where the release mechanism is diffusion governed by the porosity of the reservoir; that is, the size, distribution and connectivity of the pores. Due in part to the methods of making porous materials, controlling porosity and pore connectivity in a manner that results in a controlled release profile is difficult and unpredictable. The second method involves release of a drug that is chemically or electrochemically bonded to the device via ionic, covalent or Van der Waals bonds. Drug release rates can be changed by modulating the strength of the bond between the drug and the device or a coating on the device, however, compromises in the selection of the drug and the substrate material will likely be needed in order to achieve the degree of affinity between the drug and the device necessary to obtain the desired drug release rate.

The third method of controlling release utilizes resorbable materials, primarily polymers that degrade via hydrolysis and/or enzymatic metabolism. In this case, drug release is governed by the degree of hydrophilicity of both the polymer and the drug. Bioresorbable polymers are a preferred means of controlling drug release, because the composition of a polymer can be accurately defined and adjusted to change its degree of hydrophilicity, and thereby the rate at which moisture diffuses through the polymer matrix to release the therapeutic agent. Several methods to control the release of therapeutic agents from bioresorbable polymers have been described in the literature. For example, release can be controlled by the type of functionalization of the aromatic compound used in the bioresorbable polymer, by varying the ratio of monomers that comprise a polymer, or by modifying the density of the polymer to change the diffusion and resorption rates. Crosslinking, crystallinity, and phase separation of copolymers are alternate means of controlling diffusion. Material selection and component dimensions are also controlling factors, as are the molecular weight and architecture of the polymer. The therapeutic agent can also have varying degrees of hydrophilicity and lipid solubility that will influence both release rates and duration of physiologic effect. Many of these methods for utilizing bioresorbable polymers to control the release of therapeutic agents are instructive, but incorporating them into a device for soft tissue repair can be complex, and require the introduction of materials and/or other devices irrelevant to the primary mechanical and physiologic functions of the device.

Other methods of controlling the release of a therapeutic agent have been suggested, including co-polymers and block co-polymers with various hydrophilicity/hydrophobicity ratios, and mixtures of polymers having different release rates. Incorporating a drug into different elements of a copolymer or polymer mixture without interaction between the elements is technically difficult, as is controlling release without similar interaction. In addition, the composition of copolymers is highly dependent on the relative reactivity of the two types of cyclic monomers, and on the exact polymerization conditions used. It is hard to control the composition of polymers resulting from these types of reactions, and therefore hard to predict the polymer properties that affect drug release.

A hernia is a protrusion of a tissue, structure, or part of an organ through injured muscle tissue, or through an injured membrane by which the tissue, structure, or organ is normally contained. Surgical meshes used to reinforce tissue defects in hernia repair procedures are some of the most commonly used medical implants. Even though they are known to contribute to post-operative pain, surgical meshes are used in over 90% of hernia repairs because of the reduction in recurrence rates that have been documented with their use. Although the etiology is complex and variable, surgical meshes are known to shrink as they are encapsulated in fibrous tissue; the body's natural response to any foreign body. The resulting fibrosis, along with the tissue inflammation caused by the manipulation required to access the surgical site and place the mesh, can cause a significant amount of neuropathic pain immediately after the intra-operative anesthetic wears off. Strong medications, including anti-depressants, anti-epileptics, anti-inflammatories, and opioids are often prescribed for the first 2 to 3 days, both to treat the immediate pain and to reduce the incidence of chronic pain. After the initial post-operative period, most patients experience a reduction in pain to a level that can be managed with over-the-counter analgesics. Developments to address the pain problem have focused on reducing the mass of material comprising the mesh in order to minimize the foreign body response. One method is to reduce the number and size of the fibers comprising the mesh. A popular example of this design is the 3DMax Light Mesh from the Davol Div. of CR Bard, Inc. A similar product on the market is the Ultrapro® Partially Absorbable Lightweight Mesh from Ethicon, Inc. which is comprised of a combination of bioresorbable and non-bioresorbable fibers. In theory, the bioresorbable fibers provide additional support during the initial healing period, and then resorb leaving a light weight permanent mesh. However, clinical results for these and similar devices have not supported the design theory of less inflammatory response. This is likely due to the use of relatively slow resorbing polymers, which continue to release inflammatory degradation bi-products for a significant period of time.

Even with all of the prior art described above, there is still a need for a hernia repair mesh that contains a sufficient quantity of anesthetic to maintain an efficacious level for at least 3 days after surgery, and that releases the anesthetic into the adjacent tissue at a steady rate to avoid fluctuations in the level of pain relief. Ideally, the therapeutic effect is achieved by the mesh without adding any other materials or coatings, or otherwise altering any of the physical or mechanical properties of the mesh, or its physiologic effect on healing.

Several mesh and mesh like constructs have been proposed in the prior art to accomplish the objective of delivering an anesthetic to a local anatomical site, for example: films and coatings, of either single or multiple layers. Films are considered to be necessary by some in order to provide a depot capable of containing a sufficient quantity of drug to prolong the analgesic effect. However, films and coatings are suboptimal because they will negatively alter the physical and mechanical properties of the mesh, in terms of its elasticity and other important characteristics. Films will also inhibit tissue ingrowth by blocking the voids in the mesh construct which are necessary for the mesh to become incorporated into and support the tissue bordering the defect. Others have proposed films, coatings, fibers, mesh and the like comprised of multiple different layers or regions, or having varying densities to achieve a combination of burst and sustained release. However, the use of multiple layer constructs will not adequately meet the mechanical and physical requirements of the mesh, and will be difficult to construct in such a way that the layers are compatible and do not interact, and do not adversely affect the other desirable properties of the device.

Specific references to relevant prior art are herein described as follows:

Chefitz, US Application no. 20050015102 proposes a hernia mesh prosthesis incorporating an anesthetic having a predetermined rate of release achieved by utilizing a depot or incorporating the anesthetic within the device. This application is useful in describing the general field of the present invention, but does not contain any teachings or art related to the means of controlling release proposed in this application.

Peniston, et al, US Application no. 2013026137, describes hernia mesh composed of bioresorbable and non-bioresorbable fibers co-knit to form an interdependent mesh structure that provides an initially high level of structural stiffness. Upon degradation of the bioresorbable fibers, the non-bioresorbable fibers are “liberated” resulting in a mesh with more compliance comparable to abdominal wall tissue. This application is again useful in further outlining the general field, but in fact, teaches away from the present invention that proposes to keep the function of the bioresorbable fibers which is to contain and release bioactive and therapeutic agents, entirely separate from the function of the non-bioresorbable fibers which is to provide support to the abdominal wall. The physical and mechanical properties of the ideal mesh should match the abdominal wall tissue beginning at implantation, not some time in the future after a portion of the device has been resorbed. A mesh that changes its physical and mechanical characteristics over time will, by definition, never have the ideal structure for repairing damaged tissue.

Buevich, et al, U.S. Pat. No. 8,591,531, U.S. Pat. No. 9,023,114 disclose modulating drug release using multiple coating layers each made from the same or different polymers with varying thicknesses. However, by adding coating(s) that alter the device, Buevich essentially teaches away from the present invention which has the objective of avoiding alteration of the physical properties of the base mesh, or the incorporation of additional foreign matter.

Davis, et al, U.S. Pat. No. 8,128,954 discloses a biodegradable, drug eluting fiber or thread for local delivery of anesthetic agents. The thread may also include a “bulb” or segment of the thread with a larger diameter to deliver a sufficient amount of drug to be efficacious for an extended period of time. In addition, the thread can be comprised of different polymers. As stated, the objective of the invention is solely to deliver a drug. Fibers and threads, like suture, are subject to high recurrence rates when used in lieu of mesh to repair hernias, and because the tissues adjacent to the muscle wall defect need to be tensioned in order to close the defect, a significant amount of pain generally accompanies a sutured repair.

Sackler, et al, U.S. Pat. No. 5,747,060 describes an injectable formulation for inducing sustained regional local anesthesia in a patient comprising a substrate comprised of a local anesthetic and an effective amount of a biocompatible, biodegradable, controlled release material prolonging the release of said local anesthetic. It also proposes achieving a desired drug release rate using a mixture of polymers having different release rate so that a linear release is achieved, even though the individual polymers do not release drug at a linear rate over the same period. Injectable formulations are obviously unrelated to the art of the present invention, and in addition, mixing polymers in the same solution has the additional challenge of avoiding interaction during manufacture and during degradation in vivo.

As described in detail above, various mechanisms for delivering therapeutic drugs to surgical sites via medical implants designed for purposes other than drug delivery have been well described in the prior art. We believe that the device described in the present invention represents a significant improvement over the prior art, and accomplishes the delivery of local bioactive and therapeutic agents to a soft tissue repair site without altering the physical, mechanical, and physiologic characteristics of the ideal mesh; an important objective ignored by said prior art.

Various devices are known in the art. However, their structure and means of operation are substantially different from the present disclosure. The other inventions fail to solve all the problems taught by the present disclosure. The present invention provides for system and method of implantable devices having a variety of functionalities. In particular, the surgical mesh of the present invention has a plurality of fibers infused with therapeutic agents that can be used to improve patient outcomes in a wide array of surgeries. At least one embodiment of this invention is presented in the drawings below, and will be described in more detail herein.

### SUMMARY OF THE EMBODIMENTS

All of the shortcomings of the alternative means of achieving sustained release of a therapeutic agent without impacting other properties of a medical device, such as adding films, coatings, microspheres, and the like, or creating multilayer constructs as described in the prior art above are eliminated by incorporating the therapeutic drugs into the soft tissue repair device itself in a unique way that does not alter its other properties, and in such a way that efficacious local anesthesia can be achieved and maintained for a period of 2-7 days post implant.

The present invention provides for an implantable medical device for controlling the rate of release of a therapeutic agent comprising: at least one non-bioresorbable polymer, and/or at least one bioresorbable polymer, wherein the at least one bioresorbable polymer have different degrees of hydrophilicity; at least one therapeutic agent contained in said at least one non-bioresorbable polymers and/or said at least one bioresorbable polymers. In some embodiments, said at least one bioresorbable polymer include at least one polymer with different monomer components. In other embodiments, the present invention further comprises a mesh structure constructed out of a plurality of fibers formed from said at least one non-bioresorbable polymer and a plurality of fibers formed from said at least one bioresorbable polymer. In alternative embodiments said plurality of fibers is constructed out of at least one filaments, comprised of at least one bioresorbable polymer with different monomer components, while in other embodiments said plurality of fibers is constructed out of at least one filaments, each constructed out of at least one bioresorbable polymer with the same monomer components but in different proportions. Preferably, said therapeutic agent is at least one anesthetic, and said at least one bioresorbable polymer having different hydrophilic affinities. Even more preferably, the implantable medical device is capable of retaining and releasing the therapeutic agent for 3-10 days after being implanted in a human body, and in some embodiments this rate can be variable, but is always sufficient to maintain anesthesia for at least 2 days, preferably at least 3 days.

Further, the fibers wherein the therapeutic drugs are incorporated are combined with the fibers without therapeutic drugs in such a way that they have no effect on the mechanical, physical or physiologic functions of said device. Similarly, the fibers that do not contain therapeutic drugs do not interact with or have any effect on the drug delivery function of the soft tissue repair device.

Further, in some embodiments the mesh is composed of a plurality of fibers constructed from a single filament, and another plurality of fibers constructed out of a plurality of filaments. Both the plurality of fibers and plurality of filaments may have different diameters.

The present invention also provides for a method of manufacturing an implantable medical device comprising the steps of: solubilizing a non-bioresorbable polymer, or a bioresorbable polymer, and at least one anesthetic drug in a solvent; extruding, the solution into a at least one filaments; forming, a plurality of fibers, each fiber being constructed out of at least one of said at least one of filaments; and constructing, a surgical mesh out of said plurality of fibers. In some embodiments, said bioresorbable polymer or said non-bioresorbable polymer and said anesthetic drug are admixed and formed into filaments by melt extrusion.

In addition to the above disclosure, the present invention teaches a medical device, comprising: a plurality of fibers comprised of at least one filament constructed out of at least one non-bioresorbable polymer; a plurality of fibers comprised of at least one filament constructed out of at least one bioresorbable polymer; at least one therapeutic agent contained in said at least one non-bioresorbable polymer and/or said at least one of bioresorbable polymer, wherein said medical device is capable of releasing said therapeutic agent as a sustained rate for at least two days when implanted in a human, and is preferably capable of generating local anesthesia over said two day period.

Preferably, said at least one non-bioresorbable polymer is polypropylene; said at least one bioresorbable polymer is selected from the group consisting of: glycolide, caprolactone, and trimethylene carbonate; and said therapeutic agent is an amino amide local anesthetic. In alternative embodiments, said plurality of fibers comprised of at least one filament is constructed out of bioresorbable filaments having a variety of diameters. More preferably, said medical device is a surgical mesh, and said plurality of fibers constructed from at least one non-bioresorbable polymer and said plurality of fibers constructed from at least one bioresorbable polymer are combined in such a way that the bioresorbable fibers have no effect on the mechanical or physical properties of the medical device and said non-bioresorbable fibers do not affect the therapeutic properties of the medical device. Further, the therapeutic agent is at least one anesthetic, and said plurality of bioresorbable fibers are comprised of least one filament that will degrade in the human body.

In yet another embodiment, said plurality of fibers comprised of at least one filament constructed from at least one bioresorbable polymer and said plurality of fibers comprised of at least one filament constructed from at least one non-bioresorbable polymer are combined in such a way that their functions are independent of one another. In other embodiments, the present invention comprises a surgical mesh, comprising a plurality of fibers made from at least one non-bioresorbable polymer; a plurality of fibers made from at least one bioresorbable polymer; and at least one therapeutic agent, wherein said plurality of fibers is comprised of, a first group of fibers constructed from filaments having a diameter and incorporating a bioresorbable polymer having a hydrophilicity such that bodily fluid completely permeates said first group of fibers and releases 10-90%, preferably 20-80%, and more preferably 50-80%, of the therapeutic agent or agents incorporated in said fibers within 24 hours of being implanted in a human; a second group of fibers constructed from filaments having a diameter and incorporating a bioresorbable polymer having a hydrophilicity such that fluid completely permeates said second group of fibers and releases 10-90%, preferably 20-80%, and more preferably 50-80%, of the therapeutic agent or agents incorporated in said fibers between 24 and 48 hours of being implanted in a human; and a third group of fibers constructed from filaments having a diameter and incorporating a bioresorbable polymer having a hydrophilicity such that fluid completely permeates said third group of fibers and releases 10-90%, preferably 20-80%, and more preferably 50-80%, of the therapeutic agent or agents incorporated in said fibers between 48 and 72 hours of being implanted in a human.

Moreover, the present invention further contemplates a method of surgically repairing a soft tissue defect in a patient comprising the steps of: accessing the defect, suturing or otherwise attaching the medical device to the margins of the defect; and closing the remaining layers of tissue by conventional methods.

Bioresorbable polymers are the most common means of controlling the rate of drug release. Most resorbable polymers degrade in the body by hydrolysis, a process by which bodily fluid diffuses through the polymer breaking the chemical bonds between the monomer molecules that comprise the polymer. As fluid diffuses through the polymer, the drug is solubilized in the fluid and released to the surrounding environment. The rate of fluid uptake, and therefore the rate any drug contained within the polymer is released, can be controlled by the chemistry of the resorbable polymer characterized by its degree of hydrophilicity. The more hydrophilic a bioresorbable polymer is, the faster it will resorb in the body of a patient. Devices comprised of bioresorbable polymers with higher hydrophilicity and faster bioresorption will have higher moisture permeation rates, and will release any drug contained within said devices faster. The thickness of a flat construct or diameter of a cylindrical construct can also influence the drug release rate by controlling the time required for fluid to permeate the entire construct.

The present invention also contemplates a medical device, comprising: a combination of non-bioresorbable and bioresorbable fibers wherein the bioresorbable fibers are comprised of at least one filaments having the same or different diameters, and at least one bioresorbable polymer capable of releasing a therapeutic agent at a decreasing or increasing rate, wherein said rate is sufficient to maintain local anesthesia at a specific anatomical site for at least two days after implantation into a human.

The medical device of the present invention is comprised of multiple different polymeric components, each with the same or different hydrophilicity and bioresorption rate, and at least one therapeutic agents incorporated into said polymeric components. In certain aspects, the difference between the polymers is that their monomer components are different. In other aspects, the monomer components of the polymers are the same, but the proportion of said monomer components is different.

It is an object of the present invention to provide a surgical mesh that can alleviate pain.

It is an object of the present invention to provide a means for anesthetizing an internal area of the human body.

It is an object of the present invention to provide a surgical mesh that is partially bioresorbable, comprised of both bioresorbable and non-bioresorbable components.

It is an objective of the present invention to describe a means of delivering therapeutic drugs, such that the drug delivery function is accomplished in a unique manner that is totally independent of the other functions of the device.

In is an object of the present invention to provide a unique means of controlling the rate of delivery of a therapeutic drug without affecting the other desirable properties of the medical device in which the therapeutic drugs are incorporated.

### BRIEF DESCRIPTION OF THE DRAWINGS

FIG. 1 shows an embodiment of a plurality of individual fibers of the present invention, knitted together to form a mesh.

FIG. 2 shows an embodiment of a plurality of fibers comprised of individual filaments woven together to form a medical device comprising mesh.

FIG. 3 shows an embodiment of a plurality of individual fibers, combined in a non-woven construct to form a medical device in the form of a mesh.

FIG. 4 illustrates the difference between a fiber and the plurality of individual filaments, comprising said fiber.

FIGS. 5A-B show an embodiment of a multifilament fiber and an embodiment of a plurality of filaments comprising said multifilament fiber of the present invention.

FIGS. 5C-5D show an embodiment of a monofilament fiber of the present invention.

FIG. 6 shows an embodiment of the present invention wherein the medical device is comprised of a combination of fibers which are constructed out of at least one bioresorbable polymer, and fibers which are constructed out of at least one non-bioresorbable polymer.

FIG. 7 shows another embodiment of the present invention wherein the medical device is comprised of fibers made from different bioresorbable polymers as well as fibers made from different non-bioresorbable polymers.

FIG. 8 shows an embodiment of a medical device of the present invention wherein the filaments comprising a fiber of the present invention are made from more than one bioresorbable polymer.

FIG. 9 shows an embodiment of a medical device of the present invention wherein the filaments comprising some of the fibers are made from one bioresorbable polymer and the filaments comprising other fibers are made from another bioresorbable polymer.

FIG. 10 shows one exemplary means of combining bioresorbable fibers with non-bioresorbable fibers, using a knit construction, wherein the bioresorbable fibers are loosely incorporated utilizing large loops that will not exert any force when the medical device is stressed in situ.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

While this disclosure refers to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the spirit thereof.

Therefore, it is intended that the disclosure not be limited to the particular embodiments disclosed.

### DEFINITIONS

As used herein, the term filament refers to the smaller diameter threads from which a larger diameter fiber is spin drawn or otherwise formed.

As used herein, the term fiber means the monofilament or multifilament construct from which the mesh is made by knitting, weaving, entangling, or otherwise integrating multiple fibers into a porous fabric construct.

As defined herein, the term mesh means a porous fabric construct comprised of multiple individual fibers knitted, woven or otherwise integrated into a single construct.

The terms therapeutic agent as used herein means a drug or chemical substance that produces a positive effect on the processes of the mind and/or body.

The term polymer as used herein means a chemical entity comprised of repeating units chemically bonded together. Monomers are the molecular units which are linked together to form a polymer.

The term bioresorbable polymer means a polymer that is degraded by exposure to moisture in a process called hydrolysis, or by enzymatic metabolism in the body. The term “degradation” is defined as the process leading to the chemical cleavage of the polymer backbone, resulting in a reduction in molecular weight and mechanical strength. The rate of polymer degradation under physiological conditions is predominantly determined by the type of bonds used to link the individual repeating units together. In vivo degradation of a medical device comprised of bioresorbable polymers can take place by bulk erosion, surface dissolution, metabolic digestion, or some combination thereof. For this application, the meaning of bioresorbable is further defined to mean loss of at least 50% of the mechanical and physical properties of any device made from a bioresorbable polymer within 6 months after implantation, and complete resorption of said device within two years after implantation in a patient, in order to specifically distinguish it from devices made with bioresorbable polymers that require more than two years to fully resorb, and are therefore functionally the same as devices made with polymers generally considered by those skilled in the art as non-bioresorbable. A device is considered to be fully resorbed in vivo when it cannot be detected in tissue under histologic examination.

The term uniform release as used herein means a sustained drug release rate that maintains the desired therapeutic efficacy level for a prolonged period of time.

The term interdependent as defined herein means components of a medical device that have an effect on the same characteristics of the device.

The term independent means that the components of a medical device act separately on different characteristics of the device.

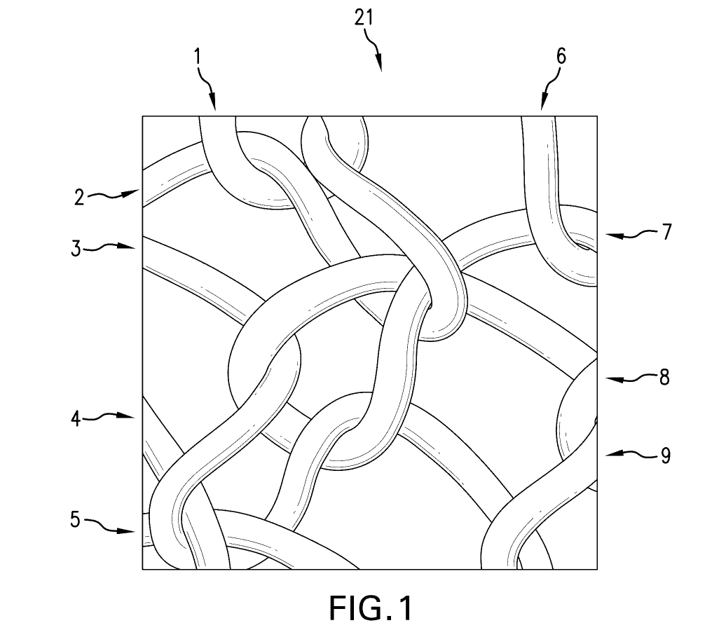

Referring to FIG. 1, a plurality of individual fibers, 1-9, knitted together to form a medical device comprising a mesh, 21 is shown. Here, the medical device may include some non-bioresorbable components in addition to the bioresorbable components. The fibers of the knitted mesh can have, for example, a stitch type of stockinette stich, reverse stockinette stitch, garter stitch, seed stitch, tricot stitch, and other patterns familiar to those skilled in the art. While here mesh 21 has been assembled by knitting a plurality of fibers, in other embodiments, the fibers may be woven or otherwise combined into a single, integrated construct. Preferably, mesh 21 is comprised of bioresorbable and non-bioresorbable polymeric fibers, some or all of which incorporate a therapeutic agent or agents. Alternatively, only the bioresorbable fibers of the medical device incorporate therapeutic agents.

One objective of the present invention is a medical device capable of delivering a therapeutic agent at a uniform rate for a sustained period of time without impacting the physical or mechanical properties of the device, or its other physiologic effects on the patient, or causing any of those properties to change over time in situ, by incorporating an efficacious quantity of said therapeutic agent directly into the device itself in a unique manner that avoids the negative aspects of the prior art as described above. To achieve this goal, many embodiments incorporate at least one therapeutic agent into at least one bioresorbable polymer components of the medical device, and using the degree of hydrophilicity or bioresorption rate of said bioresorbable polymer components to control the rate of release of said therapeutic agent. Other embodiments describe means of incorporating the bioresorbable polymer components into the medical device in such a way that the mechanical, and physical properties of said medical device are not altered.

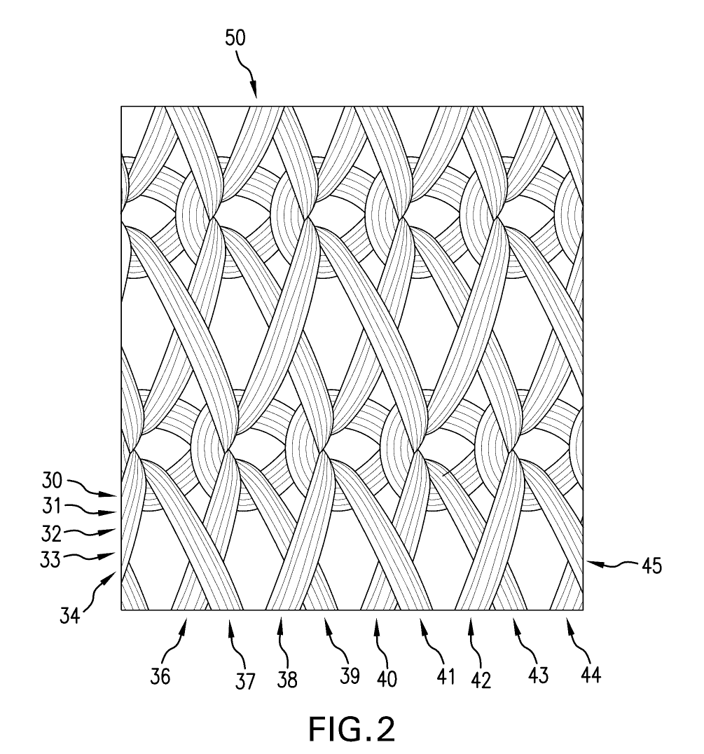

FIG. 2 is illustrative of a plurality of filaments, 30-34 constructed into individual fibers, 36-45, all woven together to form a medical device comprising mesh, 50. Preferably, this mesh, 50, will by optimized for soft tissue repair, and integrated into a construct to serve that purpose. In a variety of preferred embodiments, knitted and woven meshes are constructed by intertwining individual fibers in a series of connected loops. The physical, mechanical, and physiologic properties of the mesh are determined by the size of the fibers, the material and processes used in their construction, and the pattern in which they are knitted or woven. In some embodiments, the knit pattern is uniform such that all of the incorporated fibers are arranged in such a way that evenly distributes any forces applied by the tissue. In one embodiment of the present invention, the fibers containing the at least one therapeutic drug are incorporated into the medical device in a manner wherein said fibers experience minimal, if any, stress when forces are applied to the mesh after implantation.

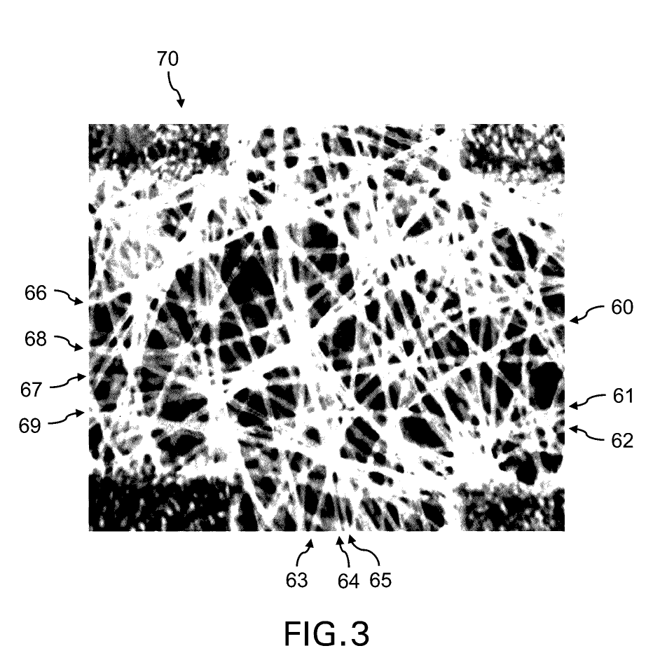

FIG. 3 shows a plurality of individual fibers, 60-69, combined in a non-woven construct to form a medical device in the form of a mesh, 70. Here, the non-woven construct serves to show that the fibers incorporated into the present invention need not be arranged in a particular order to achieve the desired therapeutic effects. While it is certainly possible for the fibers to be arranged in a pre-determined pattern, there is no need to do so, as evidenced by FIG. 3. As evidenced by this figure, the fibers used to construct mesh 70 are not limited to specific bioresorbable fibers, but rather may include some or all non-bioresorbable fibers to construct the medical device.

Further, in a preferred embodiment, the medical device is a surgical mesh generally indicated for soft tissue repair, wherein the bioresorbable polymer components are in the form of fibers used to create a mesh construct. Alternatively, the bioresorbable component may be in the form of bioresorbable filaments comprising said bioresorbable fibers. Preferably, the therapeutic agent is dissolved in the fiber during manufacture at levels defined by the solubility of a particular therapeutic agent in a particular bioresorbable polymer. Moreover, the bioresorbable polymers comprising the groups of bioresorbable filaments described above may be synthesized from the same monomers and differ from one another with respect to hydrophilicity based upon the ratio of said monomers comprising said polymers, or they may be synthesized from different base monomers, and have variable properties due to their different underlying compositions.

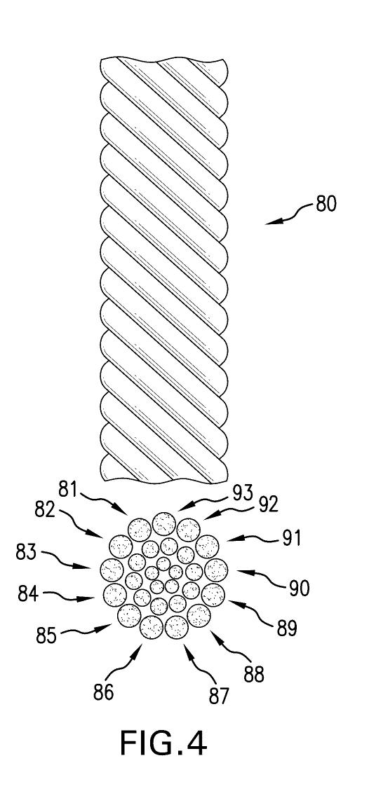

FIG. 4 illustrates the difference between a fiber, 80, and a plurality of individual filaments, 81-93, comprising said fiber, 80. As can be seen here, the individual filaments need not have the same diameter to operate within the parameters of the present invention. Rather, the different diameters allow for a given fiber to have a dramatically different resorption rate than a given fiber constructed out of differently-diametered filaments. Like the embodiment shown here, many other embodiments of the bioresorbable fibers are comprised of at least one filaments which are spin drawn or otherwise combined into said fibers. The filaments comprising a fiber may also have different diameters.

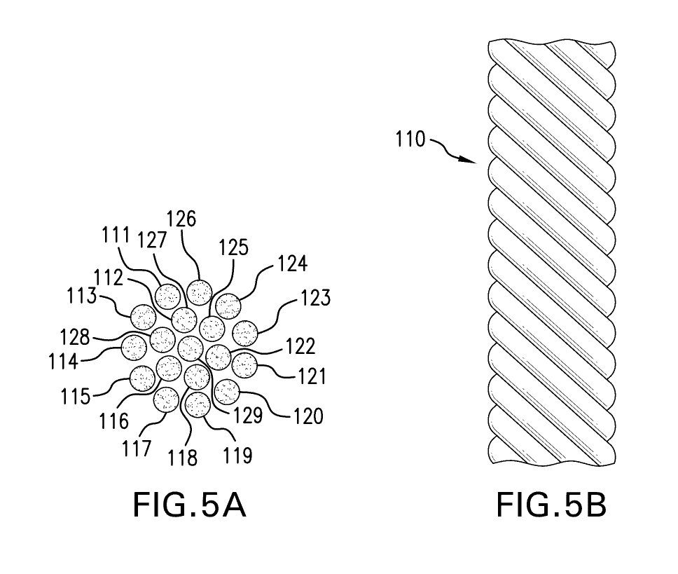

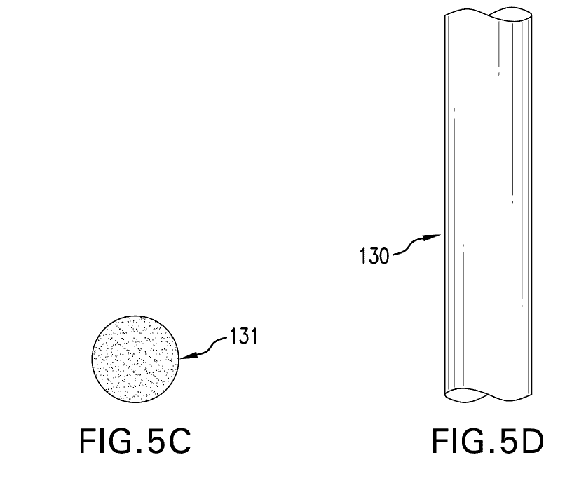

FIGS. 5A thru 5D illustrate the difference between a monofilament fiber and a multifilament fiber. FIGS. 5A and 5B show a multifilament fiber, 110, in cross-sectional view, 5A, and plan view, 5B, illustrating that said multifilament fiber, 110, is comprised of multiple filaments, 111-129. FIGS. 5C and 5D show a monofilament fiber in cross-sectional view, 5C, and plan view, 5D, illustrating that the monofilament fiber, 130, is comprised of a single filament, 131. The filaments that comprise the fibers can have varying diameters depending upon the desired mechanical and physical properties of the medical device. In yet another embodiment of the present invention, all of the bioresorbable filaments in a fiber are comprised of the same bioresorbable polymer, such that there are multiple groups of bioresorbable fibers, each group comprised of bioresorbable filaments comprised of the same bioresorbable polymer with the same degree of hydrophilicity.

In a yet another preferred embodiment of the present invention all of the non-bioresorbable fibers are comprised of medical grade polypropylene, the bioresorbable fibers are comprised of filaments synthesized from a bioresorbable polymer which is comprised of the monomers glycolide, caprolactone, and trimethylene carbonate, and the therapeutic agent is an amino amide local anesthetic. The proportion of the components of the bioresorbable polymer can vary in order to modulate the rate of anesthetic release. Preferably, the glycolide monomer component of the bioresorbable polymer comprises 50-99% of said polymer; more preferably glycolide comprises 75 to 95% of said polymer; still more preferably glycolide comprised 85-95% of said polymer. Preferably, the caprolactone component of the bioresorbable polymer comprises 1-40% of said polymer, more preferably caprolactone comprises 3-15% of said polymer, still more preferably caprolactone comprises 5-15% of said polymer.

Preferably, the trimethylene carbonate component of the bioresorbable polymer comprises 0.5-15% of said polymer, more preferably trimethylene carbonate comprises 1-8% of said polymer, still more preferably trimethylene carbonate comprises 2-5% of said polymer.

Amino amide anesthetics suitable for use with this invention include: lidocaine, bupivacaine, ropivacaine, etidocaine, prilocaine, mepivacaine and the like; more preferably lidocaine and bupivacaine; still more preferably bupivacaine. The anesthetic selected can be in any form including salt or free base. The anesthetic is incorporated into the filaments comprising the fibers comprising the mesh in a weight ratio of 5 to 90%, more preferably in a weight ratio of 10-70%, and a still more preferably in a weight ratio of 20-50%.

An example of a preferred composition of the implantable medical device of the present invention is a mesh comprised of non-bioresorbable fibers made from medical grade polypropylene, bioresorbable fibers made from a bioresorbable polymer comprised of the monomers glycolide, caprolactone and trimethylene carbonate, and the local anesthetic bupivacaine hydrochloride. The bioresorbable fibers are multifilament constructs wherein a first group of fibers is comprised of filaments, incorporating bupivacaine, which are made from said bioresorbable polymer wherein the monomers are present in the following ratio: 93-95% glycolide, 3-5% caprolactone, and 1-2% trimethylene carbonate. A second group of fibers is comprised of filaments, incorporating bupivacaine, which are made from said bioresorbable polymer wherein the monomers are present in the following ratio: 90-93% glycolide, 5-7% caprolactone, and 2-3% trimethylene carbonate. A third group of fibers is comprised of filaments, incorporating bupivacaine, which are made from said bioresorbable polymer wherein the monomers are present in the following ratio: 87-90% glycolide, 7-9% caprolactone, and 3-4% trimethylene carbonate.

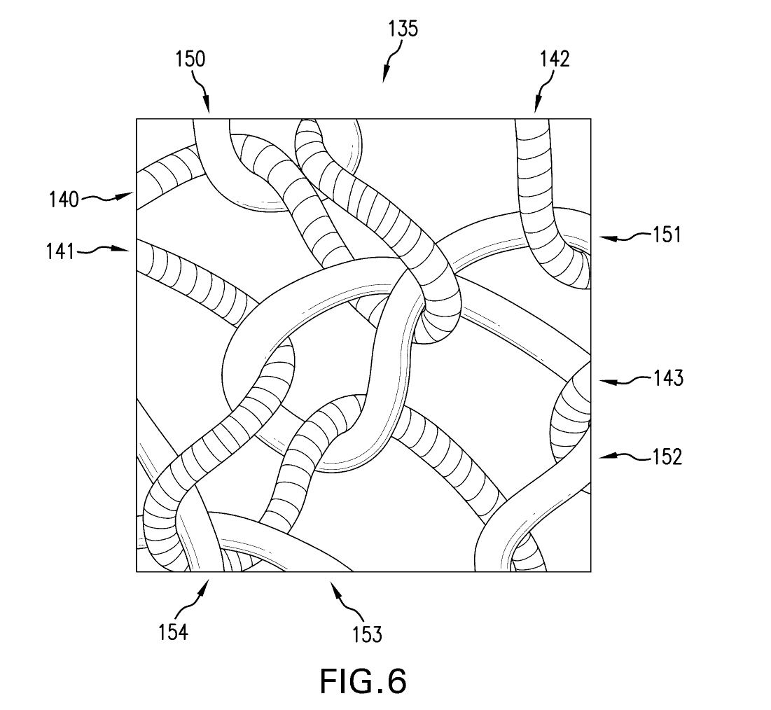

Referring to FIG. 6 an embodiment of the present invention wherein the medical device, 135, is comprised of a combination of fibers, 140-143, comprised of a bioresorbable polymer, and fibers comprised of a non-bioresorbable polymer, 150-154 is shown. This is but one configuration the present invention may take.

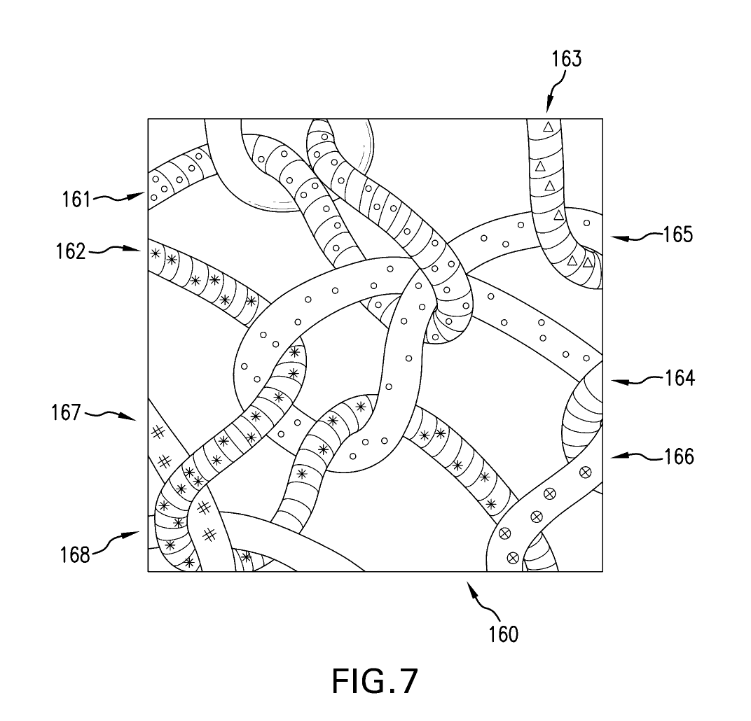

FIG. 7 shows yet another embodiment of the present invention, wherein the medical device, 160, is comprised of fibers made from different bioresorbable polymers, 161-164, and fibers made from different non-bioresorbable polymers, 165-168. Bioresorbable polymers suitable for in vivo release of therapeutic agents are selected from the following: aliphatic polyesters; polyamides; polyamines; polyalkylene oxalates; poly(anhydrides); polyamidoesters; copoly(ether-esters); poly(carbonates) including tyrosine derived carbonates; poly(hydroxyalkanoates) such as poly(hydroxybutyric acid), poly(hydroxyvaleric acid), and poly(hydroxybutyrate); (3-hydroxypropionate; polyimide carbonates, poly(imino carbonates) such as such as poly(bisphenol A-iminocarbonate) and the like; polyorthoesters; polyoxaesters including those containing amine groups; polyphosphazenes; poly (propylene fumarates); polyurethanes; dimethylsulfoniopropionate (DMSP). Also suitable are: polyester homopolymers and copolymers such as polyglycolide, polyLlactide, polyDlactide, polyD,Llactide, poly(beta-hydroxybutyrate), polyDgluconate, polyLgluconate, polyD,Lgluconate, poly(epsilon-caprolactone), poly(deltavalerolactone), poly(pdioxanone), poly(trimethylene carbonate), poly(lactidecoglycolide) (PLGA), poly(lactidecodeltavalerolactone), poly(lactidecoepsilon-caprolactone), poly(lactidecobetamalic acid), poly(lactidecotrimethylene carbonate), poly(glycolidecotrimethylene carbonate), poly(betahydroxybutyratecobetahydroxyvalerate), poly[1,3bis(pcarboxyphenoxy)propanecosebacic acid], and poly(sebacic acidcofumaric acid), among others, (b) poly(ortho esters) such as those synthesized by copolymerization of various diketene acetals and diols, among others, (c) polyanhydrides such as poly(adipic anhydride), poly(suberic anhydride), poly(sebacic anhydride), poly(dodecanedioic anhydride), poly(maleic anhydride), poly[1,3bis(pcarboxyphenoxy)methane anhydride], and poly[alpha,omegabis(p-carboxyphenoxy)alkane anhydrides] such as poly[1,3bis(pcarboxyphenoxy)propane anhydride] and poly[1,3bis(pcarboxyphenoxy)hexane anhydride], among others; and (d) amino acid based polymers including tyrosine based polyarylates (e.g., copolymers of a diphenol and a diacid linked by ester bonds, with diphenols selected, for instance, from ethyl, butyl, hexyl, octyl and bezyl esters of desaminotyrosyltyrosine and diacids selected, for instance, from succinic, glutaric, adipic, suberic and sebacic acid), tyrosinebased polycarbonates (e.g., copolymers formed by the condensation polymerization of phosgene and a diphenol selected, for instance, from ethyl, butyl, hexyl, octyl and bezyl esters of desaminotyrosyltyrosine), and tyrosine, leucine and lysinebased polyesteramides; specific examples of tyrosinebased polymers include includes polymers that are comprised of a combination of desaminotyrosyl tyrosine hexyl ester, desaminotyrosyl tyrosine, and various diacids, for example, succinic acid and adipic acid, among others.

Therapeutic agents suitable for incorporation into the medical device of the present invention include: antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Anesthetic agents suitable for incorporation into the medical device of the present invention include: acetaminophen, bupivacaine, clonidine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papavereturn, pentazocine, pethidine, phenoperidine, piritramide, dextropropoxyphene, remifentanil, tilidine, tramadol, codeine, dihydrocodeine, meptazinol, dezocine, eptazocine, and nonopioid analgesics such as flupirtine, amitriptyline, carbamazepine, gabapentin, pregabalin, or a combination thereof.

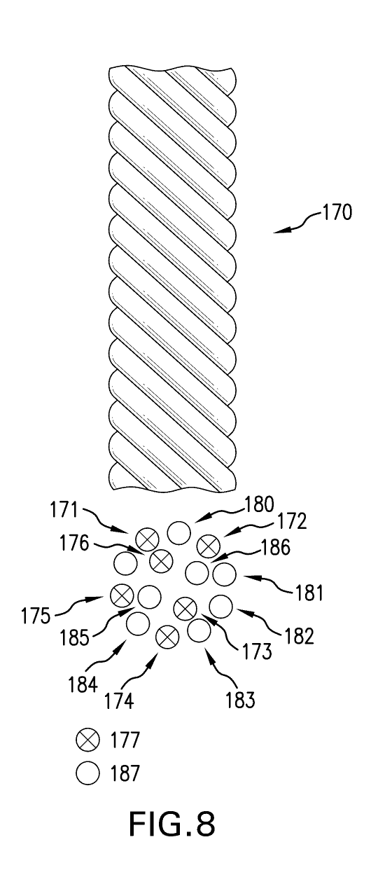

In FIG. 8 is provided a medical device of the present invention wherein the filaments comprising the fiber, 170, which comprise a device are made from more than one bioresorbable polymer. For example, filaments 171-176 are made from bioresorbable polymer, 177, and filaments, 180-186 are made from a different bioresorbable polymer, 187. One way to alter the resorption rate of the fibers, as well as the dispersion rate of an incorporated therapeutic agent is to vary the ratio of filaments comprised of different bioresorbable polymers. This is due, in part, to the inherent differences of the underlying monomers used to construct the polymers that the filaments are constructed out of. As noted above, the hydrophilicity of each polymer plays a role in the therapeutic agent disbursement process, as well as the resorption of the polymer.

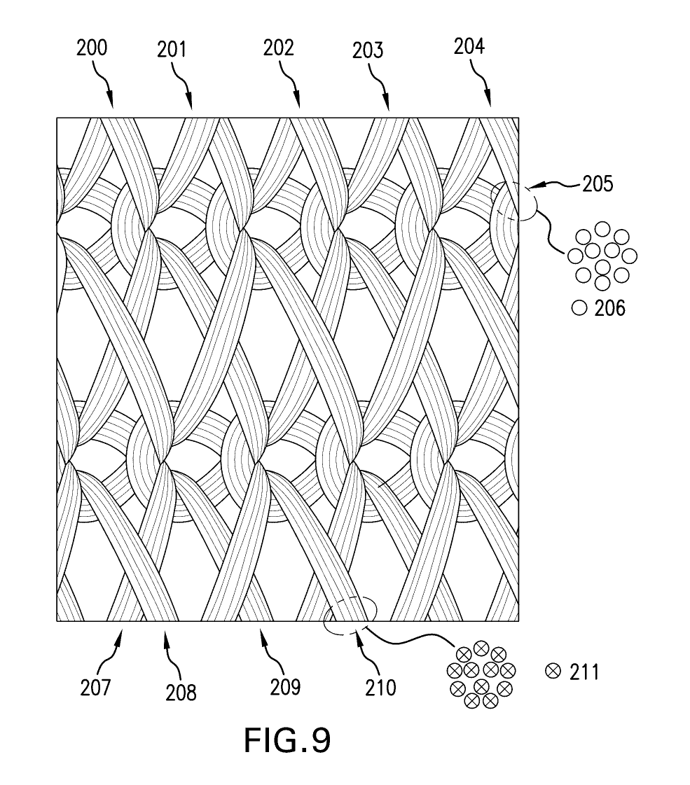

FIG. 9 shows a medical device of the present invention wherein the filaments comprising some of the fibers, 200-205, are made from one bioresorbable polymer, 206, and the filaments comprising other fibers, 207-210, are made from another bioresorbable polymer, 211.

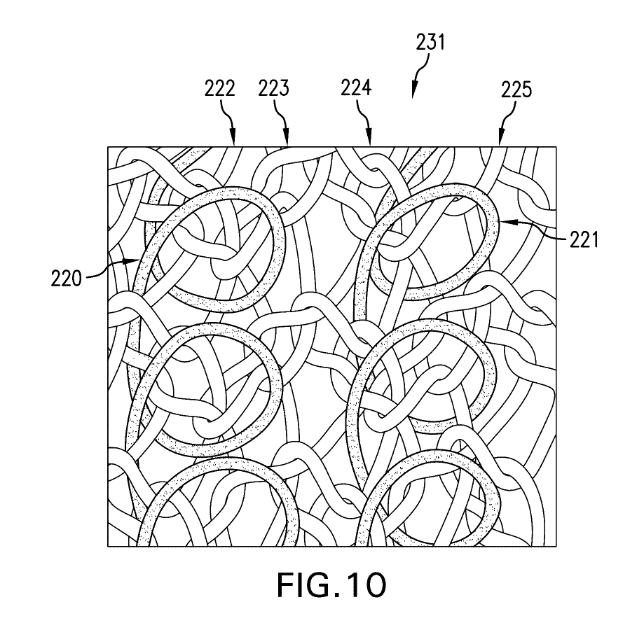

Referring to FIG. 10, one exemplary means of combining bioresorbable fibers, 220-221, with non-bioresorbable fibers, 222-225, uses a knit construction, wherein the bioresorbable fibers are loosely incorporated utilizing large loops that will not exert any force when the medical device, 231, is stressed in situ. By not being bound to a given structure, yet having the pre-defined lattice of the other fibers to operate with, a high degree of control is afforded by this configuration. In other embodiments, the fibers containing the at least one therapeutic drug are isolated from stress by knitting them into the mesh with loops that are significantly larger than the loops used to knit the fibers that do not contain the therapeutic drug(s). In another preferred embodiment of the present invention, the medical device is a surgical mesh for soft tissue repair comprising a combination of bioresorbable and non-bioresorbable fibers, wherein the bioresorbable fibers are comprised of between 1 and 50 filaments, preferably 2-25 filaments, more preferably 6-25 filaments, and most preferably 9-16 filaments.

It will be understood by those experienced in the field, that the various constructs described above are alternate means of achieving the objective of uniform release of a therapeutic agent at a level sufficient to maintain the desired pharmacologic affect for a minimum of two days, and to do so without altering the physical, mechanical and physiologic properties of a soft tissue repair device, or causing those properties to change over time in situ.

In a further preferred embodiment, the said filaments comprising said fibers are, in turn, comprised of different bioresorbable polymers with different monomer components, and as a result, different degrees of hydrophilicity. Said filaments can also vary in diameter, and as a result, the release profile of a therapeutic agent contained within some or all of the bioresorbable filaments can be adjusted by both varying the monomer components of said bioresorbable polymers, and by varying the diameters of said filaments to optimize the performance of the medical device. Preferably there are two or more groups of filaments, each with a different drug release rate. As an illustration, the bioresorbable fibers may have three different groups of bioresorbable filaments. The first group of filaments are comprised of one bioresorbable polymer and have a diameter both selected such that fluid will completely permeate said filaments and release at least 50%, preferably at least 80%, of the therapeutic agent or agents incorporated in said filaments in the period between 1 and 24 hours after implantation into a patient. The second group of bioresorbable filaments are comprised of a second bioresorbable polymer and have a diameter both selected such that fluid will completely permeate said filaments and release at least 50%, preferably at least 80%, of the therapeutic agent or agents incorporated in said filaments in the period between 24 and 48 hours after implantation into a patient. The third group of bioresorbable filaments are comprised of a third bioresorbable polymer and have a diameter both selected such that fluid will completely permeate said filaments and release at least 50%, preferably at least 80%, of the therapeutic agent or agents incorporated in said filaments in the period between 48 and 72 hours after implantation into a patient.

It will be understood by those skilled in the art that release of the therapeutic agent from each group of bioresorbable filaments is not necessarily constant over the 24 period intended for release. However, in combination with the other two groups of bioresorbable filaments, a relatively uniform release rate of said therapeutic agent can be achieved over the intended 72 hour period, such that an effective level of said therapeutic agent can be maintained for that period of time.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Therefore, it is intended that the disclosure not be limited to the particular embodiments disclosed.

What is claimed is:

1. An implantable medical device which is capable of repairing soft tissue, and means for controlling the rate of release of a therapeutic agent contained therein comprising:
at least one non-bioresorbable polymer;
a plurality of bioresorbable polymers,
wherein at least one of said plurality of bioresorbable polymers has a degree of hydrophilicity different from another of said plurality of bioresorbable polymers; and
at least one therapeutic agent contained in said plurality of bioresorbable polymers; and further comprising a mesh structure constructed from a plurality of non-bioresorbable fibers constructed from said at least one non-bioresorbable polymer, and a plurality of bioresorbable fibers constructed from said plurality of bioresorbable polymers containing said at least one therapeutic agent.

2. The implantable medical device of claim 1, wherein at least one of said plurality of bioresorbable polymers has monomer components that are different from another of said plurality of bioresorbable polymers, or has the same monomer components, but in different proportions.

3. (canceled)

4. The implantable medical device of claim 1, wherein said plurality of bioresorbable fibers are each constructed out of a plurality of bioresorbable filaments.

5. The implantable medical device of claim 1, wherein said plurality of non-bioresorbable fibers are each constructed from a single non-bioresorbable filament, and said plurality of bioresorbable fibers are each constructed from a plurality of bioresorbable filaments.

6. The implantable device of claim 1, wherein at least one of said plurality of bioresorbable fibers is comprised of a bioresorbable polymer with monomer components that are different from the monomer components in the bioresorbable polymer comprising another of said plurality of bioresorbable fibers.

7. The implantable medical device of claim 1, wherein at least one of said plurality of bioresorbable fibers is comprised of a bioresorbable polymer that is constructed out of the same monomer components as the bioresorbable polymer comprising another of said plurality of bioresorbable fibers, but in different proportions.

8. The implantable medical device of claim 4, wherein said plurality of bioresorbable fibers and said plurality of bioresorbable filaments have a range of diameters.

9. The implantable medical device of claim 1,
wherein said at least one therapeutic agent comprises at least one anesthetic.

10. The implantable medical device of claim 1, wherein said plurality of bioresorbable polymers with different degrees of hydrophilicity have different moisture resorption rates and different release rates for said at least on therapeutic agent contained therein, that give said implantable medical device the capability to retain and release said at least one therapeutic agent for 3-10 days after being implanted in a human body.

11. The implantable medical device of claim 1, wherein said plurality of bioresorbable polymers with different degrees of hydrophilicity have different moisture resorption rates and different release rates for said at least on therapeutic agent contained therein that is an at least one anesthetic which gives said implantable medical device the capability to release said at least one anesthetic at a sustained rate sufficient to maintain anesthesia for at least 2 days after implantation into the human body.

12. (canceled)

13. An implantable medical device for the treatment of post-operative pain and for the repair of soft tissue comprised of: a surgical mesh structure incorporating a plurality of non-bioresorbable fibers; and at least one anesthetic contained in a plurality of bioresorbable fibers with different degrees of hydrophilicity and lipid solubility; and wherein said implantable medical device is capable of maintaining local anesthesia for at least 2 days after implantation into the human body.

14. (canceled)

15. (canceled)

16. (canceled)

17. An implantable medical device in the form of a knitted mesh structure capable of repairing soft tissue comprising:
a plurality of non-bioresorbable fibers comprised of at least one non-bioresorbable polymer;
a plurality of bioresorbable fibers comprised of a plurality of bioresorbable polymers wherein one of said plurality of bioresorbable fibers has a hydrophilicity that is different from another of said plurality of bioresorbable fibers; and
at least one therapeutic agent contained in said of plurality bioresorbable fibers, and
wherein said implantable medical device is capable of releasing said at least one therapeutic agent at a sustained rate sufficient to maintain local therapeutic effect for at least 2 days, after implantation into the human body.

18. (canceled)

19. The medical device of claim 17, wherein said plurality of bioresorbable fibers are comprised of a plurality of bioresorbable filaments having different diameters which require different amounts of time for body fluids to completely permeate resulting in different release rates for said at least one therapeutic agent, and wherein said implantable medical device has the capability to achieve and maintain local therapeutic effect for at least 2 days after implantation into the human body.

20. The implantable medical device of claim 17, wherein said plurality of non-bioresorbable fibers in said knitted mesh structure are constructed from at least one non-bioresorbable polymer capable of integrating into soft tissue that is comprised of polypropylene.

21. The medical device of claim 17,
wherein said plurality of bioresorbable fibers comprised of said plurality of bioresorbable polymers having different degrees of hydrophilicity have different rates of moisture resorption and different release rates for said at least one therapeutic agent contained therein; wherein said implantable medical device is capable of maintaining a therapeutic effect for at least 2 days after implantation into the human body.

22. (canceled)

23. An implantable medical device comprising:
a plurality of non-bioresorbable fibers comprised of polypropylene;
a plurality of bioresorbable fibers comprised of a plurality of bioresorbable polymers composed of monomers including at least one of lactide, glycolide, caprolactone, and trimethylene carbonate; and
at least one therapeutic agent that is a incorporated within said plurality of bioresorbable fibers.

24. An implantable medical device in the form of
a surgical mesh structure, comprising:
a plurality of non-bioresorbable fibers comprised of at least one non-bioresorbable polymer that provide mechanical properties of said implantable medical device, but do not contain a therapeutic agent, and therefore do not contribute to the therapeutic effect of said implantable medical device; and
a plurality of bioresorbable fibers comprised of a plurality of bioresorbable polymers capable of containing and controlling the release of at least one therapeutic agent contained therein, and wherein said plurality of bioresorbable fibers are incorporated into said surgical mesh structure utilizing large loops that will not exert any force when said implantable medical device is stressed

25. (canceled)

26. (canceled)
